(19) United States
(12) Patent Application Publication
(10) Pub. No.: US 2015/0110851 A1
(43) Pub. Date: Apr. 23, 2015

(54) ORAL ADHERING PATCH WITH DIMPLE ON ADHERING SIDE

(71) Applicant: ORAHEALTH CORP., Bellevue, WA (US)

(72) Inventor: Jeffrey T. Haley, Mercer Island, WA (US)

(73) Assignee: ORAHEALTH CORP., Bellevue, WA (US)

(21) Appl. No.: 14/588,766

(22) Filed: Jan. 2, 2015

Related U.S. Application Data
(63) Continuation-in-part of application No. 12/287,647, filed on Oct. 10, 2008, now Pat. No. 8,945,606.
Application No. 12/287,647 is a continuation of application No. PCT/US2007/009032, filed on Apr. 13, 2007.
(60) Provisional application No. 60/792,121, filed on Apr. 13, 2006.

(51) Int. Cl. A61K 9/70; A61K 31/245; A61K 9/00; A61K 31/19

(52) U.S. Cl. CPC A61K 9/703; A61K 31/19; A61K 31/245; A61K 9/0063; A61K 9/006
USPC 424/444; 514/557; 514/535; 428/174; 428/64.1

(57) ABSTRACT

A dissolving oral adhering patch with a dimple on at least one side to improve adhesion to hard convex surfaces in the mouth, including teeth, orthodontic braces and gums (keratinized gingiva). The patch may be designed to prevent or treat a sore in the mouth opposite a tooth or brace and may release a medication into the mouth, to treat a sore or to treat or prevent conditions elsewhere in the mouth or throat or stomach.

### CROSS-REFERENCE TO RELATED APPLICATIONS

This application is a continuation-in-part of U.S. application Ser. No. 12/287,647, filed 10 Oct. 2008, which is a continuation of PCT/US2007/009032, filed 13 Apr. 2007, which claims priority of U.S. provisional application 60/792,121, filed 13 Apr. 2006, all of which are incorporated in their entirety.

### TECHNICAL FIELD

The present disclosure relates to a mucoadhering patch capable of being to be adhered to a moist surface of a mucous membrane or teeth in humans or other animals. More particularly, it relates to a patch that has a dimple on the adhering side.

### SUMMARY OF THE INVENTION

The present disclosure provides a dimpled, adhering patch, comprising, a convex surface; a concave surface opposite the convex surface; the concave surface comprising an adhesive that adheres in the mouth; wherein the patch is at least 5 mm in each of two orthogonal dimensions. In one embodiment, the oral adhering patch is approximately round and has a diameter of about 9 millimeters. In another embodiment, the patch erodes when held in a human mouth. In another embodiment, the patch further comprises an active ingredient, such as glycyrrhetinic acid or benzocaine. In certain embodiments, the concave surface has a depth of at least 0.5 millimeters. In yet other embodiments, the adhesive comprises gelatin, acacia gum or collagen.

### BRIEF DESCRIPTION OF THE DRAWINGS

FIGS. 1A-B show a side view (A) and a top view (B) of an exemplary oral patch.

FIG. 2 shows an exemplary domed oral patch with a dimple.

### DETAILED DESCRIPTION

To deliver an active ingredient, such as a medication in the mouth over time for treatment of health problems in the mouth or throat, oral patches have been developed. The present disclosure provides an oral adhering disc with a dimple. The disc, which may also be referred to as a patch or troche, comprises a first surface that is concave and is capable of adhering to a surface in a mouth of a human or animal. Opposite the first surface, the disc comprises a second surface, which may be convex or essentially flat. The patch may comprise an active ingredient, such as a medicament. Adhered to a surface in the mouth, the patch erodes over time.

As used herein, the term “adhering oral patch (or troche or disc)” refers to an adhesive device that is capable of sticking to a mucosal or otherwise wet surface in a mouth of a human or other animal. In certain embodiments, the patch can adhere to gums or teeth, structures attached to teeth (e.g., orthodontic braces), or oral mucosal tissue. The oral patch as disclosed herein is designed to release one or more active ingredients (e.g., medication) into the mouth over a relatively long period of time, such as 30 minutes or more. Because it is adherent, staying in one place, and erodes in place, the active ingredient can reach high concentrations in the vicinity of the patch. The patch is manufactured to gradually erode or wear away or dissolve as a single item, rather than crumble or disintegrate into many pieces.

An adhering oral patch may be any shape, including round (when viewed from above or below), rectangular, ovoid, other polygonal shape, or irregularly shaped. The shape is chosen at least in part by the manufacturing process. Typically, the length of orthogonal dimensions is in the range of about 5 to about 18 mm, and the thickness from about 1 mm to about 10 mm. In some embodiments, the patch may have a dimension as large as 30 mm. One of the surfaces is concave. The opposite surface can be flat, essentially flat, convex, domed, or irregular. In certain embodiments, the two surfaces have different shapes, such as one side being convex and the other side having a dimple (concave). The size of the nonadherent layer can be larger than the adherent layer, such that the surface of the adherent layer is e.g. “ringed” by the nonadherent layer, as long as the patch is able to adhere to a mucosal surface.

A suitable shape for adhering to a tooth or braces that caused a cut that has become or is likely to become an ulcer is a dimpled dome—that is, convex on one side and concave on the other side. An example is shown in cross-section in FIG. 2. The dimple may be a slight concavity. Nine millimeters diameter is an exemplary size for such a dimpled dome made by pressing powders, with 0.5 to 1.5 millimeters for the depth of the dimple. For adhering to a bracket of orthodontic braces, such a dimple will allow greater contact with the bracket and wires for better adhesion. For adhering to a tooth, the concave dimple will allow the patch to adhere to a convex tooth surface at the periphery of the patch with multiple points of contact rather than with essentially a single point of contact near the center of a flat or convex patch surface.

A detailed description of a deposited patch and how to make it are disclosed by the same inventor in U.S. patent application Ser. No. 10/287,843 filed 5 Nov. 2002 and incorporated herein in its entirety. A detailed description of a pressed powders patch made with mucoadhesive hydrocolloids pressed in two layers, one quite adhesive, is disclosed by the same inventor in U.S. patent application Ser. No. 11/800,381 filed 4 May 2007 and incorporated herein in its entirety.

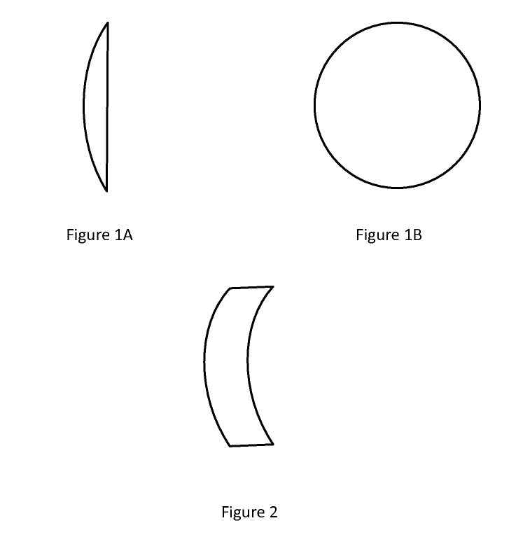

An exemplary method of manufacturing the patches of FIG. 1 is to use gum drop manufacturing equipment, squirting a hydrated mixture heated above the gel melting temperature through nozzles onto a sheet of plastic or mold, allowing the patches to cool and gel, and drying the patches. The patches are typically dried until the water activity level is lower than 0.8 so that the patches will not grow mold or other organisms. The patches are packaged with a hermetic seal to prevent absorption of water moisture from air. The resulting patches are typically at least 5 mm in each of at least two dimensions, preferably 8-18 mm.

An oral adhering patch may comprise other ingredients. In some embodiments, a patch comprises an active ingredient, such as a medicament. Active ingredients can be any substance that treats or ameliorates a condition or disease, that prevents a condition or disease, or that provides a pleasurable experience for the user (e.g., a flavor), etc. Other types of ingredients include for example, one or more adhesives (e.g., acacia gum), one or more binders (e.g., cellulose gum, acacia gum), one or more flavorings (e.g., licorice), one or more vitamins, one or more coloring agents, one or more corrigents, one or more flavors, one or more excipients (e.g., salts, buffers), one or more viscous and lubricating substances (e.g., cellulose gum), and one or more adjuvants. Other ingredients may also include one or more lubricants or one or more glidants. In some embodiments only one additional ingredient may be present, in other embodiments, multiple other ingredients may be present. Compositions of other ingredients are well-known in the art.

The patch may comprise one or more binders. The binder ingredients may be a combination of gums that dissolve in saliva, and non-dissolving food fibers. Some suitable binders include carrageenan (especially kappa form), xanthan gum, xanthan gum combined with konjac gum, agar, and cellulose gums such as carboxymethylcellulose (CMC), hydroxyproplycellulose (HPC) and hydroxyproplymethylcellulose (HPMC). Other gums similar to those listed, such as locust bean gum which has properties similar to konjac gum, and guar gum are also suitable, as well as starches, such as corn starch or, particularly pregelatinized corn starch. In addition, the binder may also moderate any strong flavors by spreading out over a long period of time the release of that flavor. Consequently, sweeteners and other products to mask strong flavors are not required, although some users may prefer a small amount of sweetener and some may prefer the addition of other flavors.

When the binders are xanthan gum, konjac gum, and cellulose fiber, effective dry weight formulations include between 1% and 10% active ingredient, such as potassium salt of glycyrrhetinic acid (GTA), between 20% and 99% food grade gelatin, and between 0% and 75% other binders. Another effective formula comprises 2-4% SGE (soluble glycyrrhetinic extract) with about 5-7% benzocaine and 50-93% gelatin, with acacia gum added on the side intended to be adherent. (GTA) is a mer component of glycyrrhizic acid, which is the negative part of the salt glycyrrhizin, which is a major ingredient in simple water extract of licorice root.

The patch comprises an adhesive. The adherent side of the patch comprises one or more natural or synthetic polymers that have adhesiveness to the wet mucous surface or a combination of said polymers and, in some embodiments, an active ingredient, such as a medicament. Adhesive molecules include acacia gum, gelatin, alginate, starch, pectin, polyvinylpyrolidone, carboxymethylcellulose, hydroxymethylcellulose, polyvinyl acid, polyacrylic acid, and carbopol. Concentrations of these adhesive molecules are well-known. For example, one embodiment provides a hydrocolloid that holds together as a single item when held in the mouth, such as the adherent, soluble oral patch disclosed by the same inventor in U.S. patent application Ser. No. 10/287,843 filed Nov. 5, 2002, and incorporated herein in its entirety.

For an oral patch made by a tablet pressing process, the typical size is about 100 to 150 milligrams for total tablet weight. An exemplary patch formulation is made by combining the active ingredient with collagen and with binder ingredients. Collagen, which is the organic molecule that makes up skin and the lining of the mouth (a form of skin), tends to adhere very well to itself, making it glutinous, and therefore adheres very well to the lining of the mouth. An effective and cost effective form of collagen is food grade gelatin which is made from animal skins. As the collagen molecules slough off the patch while it slowly dissolves (erodes), they tend to adhere to the nearby mouth lining, forming a film. This film significantly reduces the sensitivity of the ulcer, both to touch and to chemical irritants.

Presented below are conclusions from testing on 49 subjects of the adherent, soluble oral patches with about 7-9% GTA:

Pain relief: Using a patch for 10-15 minutes before a meal reduces pain of the canker sore, and, if used up to commencement of a meal, the pain relief lasts through a typical meal. There is no numbing effect on surrounding tissues.

Catching it early: If the user catches the canker sore early, shorter treatment is required. The sore will often start in a small cut. Some users report that if they apply one patch to a cut for 1-4 hours before there is any sensation of a canker sore, then they will not get a canker sore from the cut. Other times, the sore starts with a feeling that the mucous layer is becoming too thin in a spot before it becomes painful. Some users report that if they apply one patch to that spot, no canker sore develops. Users report that if they begin applying the patch when the canker sore is very small and barely painful, the patches control the pain to the extent that there is no significant pain and healing is accelerated.

Treatment of the tongue: For treatment of the tongue, most users stick a patch (which releases extract on both sides) to the closest tooth. This works particularly well at night.

Braces: Users with braces apply the patch to the braces opposite the canker sore so that the patch is touching the canker sore most of the time and is stuck to the teeth and braces. As it softens, the patch settles into the braces. It will completely dissolve out of the braces in 3-9 hours. All this time it supplies GTA to the sore.

While particular embodiments of the invention have been described above the scope of the invention should not be limited by the above descriptions but rather limited only by the following claims.

I claim:

1. A dimpled, adhering patch, comprising:
a. a convex surface;
b. a concave surface opposite the convex surface;
c. the concave surface comprising an adhesive that adheres in the mouth;
wherein the patch is at least 5 mm in each of two orthogonal dimensions.

2. The oral adhering patch of claim 1, being approximately round and having a diameter of 8-18 mm.

3. The oral adhering patch of claim 1, wherein the patch erodes when held in a human mouth.

4. The oral adhering patch of claim 1, further comprising an active ingredient.

5. The oral adhering patch of claim 4, wherein the active ingredient comprises glycyrrhetinic acid.

6. The oral adhering patch of claim 4, wherein the active ingredient comprises benzocaine.

7. The oral adhering patch of claim 1, wherein the concave surface has a depth of at least 0.5 millimeters.

8. The oral adhering patch of claim 1, wherein the adhesive comprises gelatin, acacia gum or collagen.
